(19) United States
(12) Patent Application Publication
(10) Pub. No.: US 2018/0057867 A1
(43) Pub. Date: Mar. 1, 2018

(54) Methods of Hybridizing Probes to Genomic DNA

(71) Applicant: President and Fellows of Harvard College, Cambridge, MA (US)

(72) Inventors: Chao-ting Wu, Brookline, MA (US); Reza Kalhor, East Boston, MA (US)

(21) Appl. No.: 15/800,291

(22) Filed: Nov. 1, 2017

Related U.S. Application Data
(63) Continuation of application No. 14/204,429, filed on Mar. 11, 2014, now abandoned.
(60) Provisional application No. 61/781,282, filed on Mar. 14, 2013.

(51) Int. Cl. C12Q 1/68

(52) U.S. Cl. CPC C12Q 1/6832; C12Q 2543/10; C12Q 2537/162

(57) ABSTRACT

The present invention relates to methods of hybridizing nucleic acid probes to genomic DNA.

### RELATED APPLICATION DATA

This application is a continuation application which claims priority to U.S. patent application Ser. No. 14/204,429, filed on Mar. 11, 2014, U.S. provisional application 61/781,282 filed Mar. 14, 2013 each of which are hereby incorporated by reference in their entireties.

### STATEMENT OF GOVERNMENT INTERESTS

This invention was made with government support under grant number 1 RO1 GM085169 awarded by NIH. The government has certain rights in the invention.

### FIELD

The present invention relates in general to the use of oligonucleotide probes to hybridize to double stranded nucleic acids, for example, the DNA in a chromosome. The oligonucleotide probes include a labeled probe which binds to one strand of genomic DNA and one or more anti-lock probes which bind to the complementary strand of the genomic DNA. Use of the anti-lock probes improves binding efficiency of the labeled probe because the anti-lock probe inhibits re-annealing of the genomic DNA.

### BACKGROUND

Fluorescence in situ hybridization (FISH) is a powerful technology wherein nucleic acids are targeted by fluorescently labeled probes and then visualized via microscopy. FISH is a single-cell assay, making it especially powerful for the detection of rare events that might be otherwise lost in mixed or asynchronous populations of cells. In addition, because FISH is applied to fixed cell or tissue samples, it can reveal the positioning of chromosomes relative to nuclear, cytoplasmic, and even tissue structures, especially when applied in conjunction with immunofluorescent targeting of cellular components. FISH can also be used to visualize RNA, making it possible for researchers to simultaneously assess gene expression, chromosome position, and protein localization.

Labeled probes in FISH methods bind to a portion of genomic DNA that has separated into two strands. The labeled probe binds to one of the strands. However, re-annealing of the two strands can prevent the labeled probe from binding to the genomic DNA or can displace the bound labeled probe, thereby lowering the labeled probe's binding efficiency to the genomic DNA. Therefore, methods of improving binding efficiency of labeled probes to genomic DNA are desirable.

### SUMMARY

Embodiments of the present disclosure are directed to methods of improving binding efficiency of a labeled nucleic acid probe to genomic DNA, such as a genomic locus, such as DNA in a chromosome, having a portion of the genomic DNA separated into single stranded segments, such as two single stranded segments. According to certain aspects, one or more additional nucleic acid probes are used to bind to the genomic DNA in a manner to inhibit or prevent the re-annealing of the single stranded segments of the genomic DNA. The one or more additional nucleic acid probes may be referred to herein as “anti-lock” probes or “blocking” probes to the extent that they inhibit or prevent the re-annealing of the two single strand segments of the genomic DNA when they are hybridized thereto. In this manner, the efficiency of the binding of the labeled probe is increased because the anti-lock probe inhibits re-annealing which can prevent hybridization of the labeled probe or can displace a bound labeled probe.

According to certain aspects, a method of improving binding efficiency of a labeled probe to double stranded DNA having a portion of the double stranded DNA separated into a first single strand segment and a complementary single strand segment is provided which includes combining the double stranded DNA with a labeled probe that is complementary to the first single stranded segment and one or more anti-lock probes that are complementary to either the first single stranded segment or the complementary single stranded segment wherein the labeled probe binds to the first single stranded segment and the one or more anti-lock probes bind to at least the complementary single stranded segment. According to one aspect, the double stranded DNA is genomic DNA. According to one aspect, the bound one or more anti-lock probes inhibits re-annealing of the first single strand segment and the complementary single strand segment.

According to one aspect, the labeled probe is between 2 nucleotides and 200 nucleotides in length. According to one aspect, the labeled probe is an oligonucleotide paint as described in US 2010/0304994.

According to one aspect, one or more anti-lock probes binds to the complementary single stranded segment at a position which neighbors or overlaps with the region in the genomic DNA that is complementary to the target sequence of the labeled probe.

According to one aspect, a first anti-lock probe binds to the complementary single stranded segment at a position which neighbors or overlaps with the region in the genomic DNA that is complementary to the target sequence of the labeled probe. According to one aspect, a first anti-lock probe binds to the complementary single stranded segment at a position which overlaps with the region complementary to the target sequence of the labeled probe by at least 1 nucleotide. According to one aspect, a first anti-lock probe binds to the complementary single stranded segment at a position which overlaps with the region complementary to the target sequence of the labeled probe by between about 1 nucleotide and about 10 nucleotides. According to one aspect, a first anti-lock probe binds to the complementary single stranded segment at a position which overlaps with the region complementary to the target sequence of the labeled probe by between about 1 nucleotide and about 5 nucleotides.

According to one aspect, a first anti-lock probe binds to the complementary single strand segment at a position which overlaps with the region complementary to the target sequence of the bound labeled probe and a second anti-lock probe binds to the complementary single stranded segment at a position which overlaps with the region complementary to the target sequence of the bound labeled probe by at least one nucleotide. According to one aspect, a first anti-lock probe binds to the complementary single strand segment at a position which overlaps with the region complementary to the target sequence of the bound labeled probe and a second anti-lock probe binds to the complementary single strand segment at a position which overlaps with the region complementary to the target sequence of the bound labeled probe by between about 1 nucleotide and about 10 nucleotides.

According to one aspect, a first anti-lock probe binds to the complementary single stranded segment at a position which overlaps with the region complementary to the target sequence of the bound labeled probe and a second anti-lock probe binds to the first single strand segment at a position which overlaps with the region complementary to the target sequence of the first antilock probe. According to one aspect, a first anti-lock probe binds to the complementary single stranded segment at a position which overlaps with the region complementary to the target sequence of the bound labeled probe and a second anti-lock probe binds to the first single strand segment at a position which overlaps with the region complementary to the target sequence of the first antilock probe by between about 1 nucleotide and about 10 nucleotides. According to one aspect, a first anti-lock probe binds to the complementary single stranded segment at a position which overlaps with the region complementary to the target sequence of the bound labeled probe and a second anti-lock probe binds to the first single strand segment at a position which overlaps with the region complementary to the target sequence of the first antilock probe by between about 1 nucleotide and about 5 nucleotides.

According to one aspect, the labeled probe and two or more anti-lock probes are connected. According to one aspect, the labeled probe and the two or more anti-lock probes are connected by one or more connector nucleotides. According to one aspect, the labeled probe and the two or more anti-lock probes are connected in series by one or more connector nucleotides to form a continuous oligonucleotide strand. According to one aspect, the labeled probe and the two or more anti-lock probes are connected in series by one or more connector nucleotides to form a continuous oligonucleotide strand with the labeled probe being at one end of the continuous oligonucleotide strand. According to one aspect, the labeled probe and the one or more anti-lock probes are connected in series by one or more connector nucleotides to form a continuous oligonucleotide strand with the labeled probe being at one end of the continuous oligonucleotide strand and with a first anti-lock probe being hybridized to the first single strand segment. According to one aspect, the labeled probe and the one or more anti-lock probes are connected in series by one or more connector nucleotides to form a continuous oligonucleotide strand with the labeled probe being at one end of the continuous oligonucleotide strand or between two anti-lock probes and with a first anti-lock probe being hybridized to the complementary single strand segment. According to one aspect, the labeled probe and the one or more anti-lock probes are connected in series by one or more connector nucleotides to form a continuous oligonucleotide strand with the labeled probe being at one end of the continuous oligonucleotide strand or between two anti-lock probes and with a first anti-lock probe being hybridized to the first single strand segment and a second antilock probe being hybridized to the complementary single strand segment.

According to one aspect, the labeled probe and the one or more anti-lock probes are connected by one or more connector nucleotides wherein the one or more connector nucleotides are unhybridizable to the first single strand segment or the complementary single strand segment. According to one aspect, the labeled probe and the one or more anti-lock probes are connected by linker portions.

According to one aspect, the labeled probe and the one or more anti-lock probes include one or more of self-avoiding nucleotide analogues. According to one aspect, the labeled probe and the one or more anti-lock probes include one or more of self-avoiding nucleotide analogues such that the labeled probe and the one or more anti-lock probes do not hybridize to each other. According to one aspect, the labeled probe and a first anti-lock probe include one or more of self-avoiding nucleotide analogues such that the labeled probe and the one or more anti-lock probes are complementary sequences and do not hybridize to each other.

According to one aspect, the labeled probes and the antilock probes are hybridized at the same time. According to one aspect, the one or more anti-lock probes are hybridized to the single stranded DNA followed by hybridization of the labeled probe to the complementary single stranded DNA.

According to one aspect, the term labeled probe refers to both a single molecule including a probe sequence and a label attached thereto, such as by covalent attachment, or a probe sequence and a separate label component which are added as separate species but then combine to form a labeled probe. Such an embodiment may be referred to as a secondary label. Accordingly, when reference is made to “combining the double stranded DNA with a labeled probe,” such combining step includes the probe and the label being separate components being added to a double stranded nucleic acid, and then combining to form a labeled probe at some point during the method which is hybridized to a single strand portion of the double stranded nucleic acid.

According to one aspect, certain nucleic acid probes may be labeled or unlabeled. Certain nucleic acid probes may be directly labeled or indirectly labeled. According to certain aspects, nucleic acid probes may include a primary nucleic acid sequence that is non-hybridizable to a target nucleic acid sequence. According to certain aspects, the primary nucleic acid sequence is hybridizable with a secondary nucleic acid sequence. According to certain aspects, the secondary nucleic acid sequence may include a label. According to this aspect, the nucleic acid probes are indirectly labeled as the secondary nucleic acid binds to the primary nucleic acid thereby indirectly labeling the probe which hybridizes to the target nucleic acid sequence. According to certain aspects, the secondary nucleic acid sequence hybridizes with the primary nucleic acid sequence to create a recognition sequence which may be recognized or bound by a functional moiety. According to certain aspects, a plurality of nucleic acid probes are provided with each having a common primary nucleic acid sequence. That is, the primary nucleic acid sequence is common to a plurality of nucleic acid probes, such that each nucleic acid probe in the plurality has the same or substantially similar primary nucleic acid sequence. In this manner, a plurality of common secondary nucleic acid sequences are provided which hybridize to the plurality of common primary nucleic acid sequences. That is, each secondary nucleic acid sequence has the same or substantially similar nucleic acid sequence. According to one exemplary embodiment, a single primary nucleic acid sequence is provided for each of the nucleic acid probes in the plurality. Accordingly, only a single secondary nucleic acid sequence which is hybridizable to the primary nucleic acid sequence need be provided to label each of the nucleic acid probes. According to certain aspects, the common secondary nucleic acid sequences may include a common label. According to this aspect, a plurality of nucleic acid probes are provided having substantially diverse nucleic acid sequences hybridizable to different target nucleic acid sequences and where the plurality of nucleic acid probes have common primary nucleic acid sequences. Accordingly, a common secondary nucleic acid sequence having a label may be used to indirectly label each of the plurality of nucleic acid probes. According to this aspect, a single or common primary nucleic acid sequence and secondary nucleic acid sequence pair can be used to indirectly label diverse nucleic acid probe sequences. Methods using nucleic acid probes as described herein include any method where probe hybridization is useful, including but not limited to fluorescence in situ hybridization methods known to those of skill in the art or any other method where a label, such as a functional moiety, is desired to be brought to or near a target nucleic acid sequence through hybridization of the probe to the target nucleic acid sequence for detection, chemical modification, retrieving or binding to a target molecule, or providing other functions.

### BRIEF DESCRIPTION OF THE DRAWINGS

The foregoing and other features and advantages of the present invention will be more fully understood from the following detailed description of illustrative embodiments taken in conjunction with the accompanying drawing in which:

FIG. 1 is a schematic representation of standard hybridization conditions without anti-lock probes using a labeled probe where a low percentage of the probe molecules hybridize to the target genomic DNA (gDNA) and a high percentage of the probe molecules remain unhybridized.

FIG. 2 is a schematic representation of hybridization using a labeled probe (brown) and two anti-lock probes (blue) where the binding of the anti-lock probes prevents the re-annealing of the target genomic DNA, resulting in a higher percentage of the labeled probe being hybridized.

FIG. 3 is a schematic representation showing partial overlap of anti-lock probes with a labeled probe.

FIG. 4 is a schematic representation of hybridization of a nucleic acid sequence including a labeled probe portion and two anti-lock probe portions that are combined into a single molecule using connectors (black lines).

FIG. 5 is a schematic representation of hybridization of two separate nucleic acid sequences with each including a labeled probe portion and two anti-lock probe portions.

FIG. 6 is a schematic representation of hybridization of a labeled probe and an anti-lock probe with each including self-avoiding nucleotides and being complementary to each other.

FIG. 7 depicts exemplary self-avoiding nucleotides.

### DETAILED DESCRIPTION

Terms and symbols of nucleic acid chemistry, biochemistry, genetics, and molecular biology used herein follow those of standard treatises and texts in the field, e.g., Komberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

According to embodiments of the present disclosure, a method of improving binding efficiency of a labeled nucleic acid probe to genomic DNA, such as a genomic locus, having a portion of the genomic DNA separated into two single strand segments. According to certain aspects, “anti-lock” probes or “blocking” probes are used to bind to the genomic DNA in a manner to inhibit or prevent the re-annealing of the two single strand segments of the genomic DNA. Since the two separate strands are inhibited from re-annealing, the labeled probe more efficiently binds to the genomic DNA.

Methods according to the present disclosure include any methods known to those of skill in the art where nucleic acid probes are used to hybridize to double stranded DNA where a portion of the double stranded DNA has separated into two separate strands, i.e. a first strand and a complementary strand. It is to be understood that reference to a first strand and a complementary strand is relative when separating double stranded nucleic acids. That is, either strand can be the first strand or the complementary strand. Selecting one strand as the first strand makes the remaining strand the complementary strand.

One exemplary method where the labeled probes and the anti-lock probes described herein have particular utility include fluorescent in situ hybridization or FISH which is a cytogenetic technique that is used to detect and localize the presence or absence of specific DNA sequences on chromosomes. FISH uses fluorescent probes that bind to only those parts of the chromosome with which they show a high degree of sequence complementarity. Fluorescence microscopy can be used to find out where the fluorescent probe is bound to the chromosomes. FISH is often used for finding specific features in DNA for use in genetic counseling, medicine, and species identification. FISH can also be used to detect and localize specific RNA targets (mRNA, lncRNA and miRNA) in cells, circulating tumor cells, and tissue samples. In this context, it can help define the spatial-temporal patterns of gene expression within cells and tissues. Exemplary FISH methods are known to those of skill in the art and are readily available in the published literature.

As used herein, the term “chromosome” refers to the support for the genes carrying heredity in a living cell, including DNA, protein, RNA and other associated factors. There exists a conventional international system for identifying and numbering the chromosomes of the human genome. The size of an individual chromosome may vary within a multi-chromosomal genome and from one genome to another. A chromosome can be obtained from any species. A chromosome can be obtained from an adult subject, a juvenile subject, an infant subject, from an unborn subject (e.g., from a fetus, e.g., via prenatal test such as amniocentesis, chorionic villus sampling, and the like or directly from the fetus, e.g., during a fetal surgery) from a biological sample (e.g., a biological tissue, fluid or cells (e.g., sputum, blood, blood cells, tissue or fine needle biopsy samples, urine, cerebrospinal fluid, peritoneal fluid, and pleural fluid, or cells therefrom) or from a cell culture sample (e.g., primary cells, immortalized cells, partially immortalized cells or the like). In certain exemplary embodiments, one or more chromosomes can be obtained from one or more genera including, but not limited to, Homo, Drosophila, Caenorhabiditis, Danio, Cyprinus, Equus, Canis, Ovis, Ocorynchus, Salmo, Bos, Sus, Gallus, Solanum, Triticum, Oryza, Zea, Hordeum, Musa, Avena, Populus, Brassica, Saccharum and the like.

Probes included within the scope of the present disclosure include those known to be useful with FISH methods. FISH probes are typically derived from genomic inserts subcloned into vectors such as plasmids, cosmids, and bacterial artificial chromosomes (BACs), or from flow-sorted chromosomes. These inserts and chromosomes can be used to produce probes labeled directly via nick translation or PCR in the presence of fluorophore-conjugated nucleotides or probes labeled indirectly with nucleotide-conjugated haptens, such as biotin and digoxigenin, which can be visualized with secondary detection reagents. Probe DNA is often fragmented into about 150-250 bp pieces to facilitate its penetration into fixed cells and tissues. As many genomic clones contain highly repetitive sequences, such as SINE and Alu elements, hybridization often needs to be performed in the presence of unlabeled repetitive DNA to prevent off-target hybridizations that increase background signal. Such probes may be referred to as “chromosome paints” which refers to detectably labeled polynucleotides that have sequences complementary to DNA sequences from a particular chromosome or sub-chromosomal region of a particular chromosome. Chromosome paints that are commercially available are derived from fluorescence activated cell sorted (FACS) and/or flow sorted chromosomes or from bacterial artificial chromosomes (BACs) or yeast artificial chromosomes (YACs).

There are several limitations to clone-based FISH probes. The genomic regions that can be visualized by these probes are restricted by the availability of the clones that will serve as templates for probe production and the size of their genomic inserts, which typically range from 50-300 kb. While it is possible to target larger regions and establish banding patterns by combining probes, this approach is labor intensive and often technically difficult, as each clone needs to be amplified, purified, labeled, and optimized for hybridization separately. The hybridization efficiency of these probes is also highly variable, even among different preparations of the same probe. This variation may be a consequence of the random labeling and fragmentation steps used during probe production.

Many types of custom-synthesized oligonucleotides (oligos) have also been used as FISH probes, including DNA, peptide nucleic acid (PNA), and locked nucleic acid (LNA) oligos. One advantage of oligo probes is that they are designed to target a precisely defined sequence rather than relying on the isolation of a clone that is specific for the desired genomic target. Also, as these probes are typically short (about 20-50 bp) and single-stranded by nature, they efficiently diffuse into fixed cells and tissues and are unhindered by competitive hybridization between complimentary probe fragments. Recently developed methods utilizing oligo probes have allowed the visualization of single-copy viral DNA as well as individual mRNA molecules using branched DNA signal amplification or a few dozen short oligo probes and, by targeting contiguous blocks of highly repetitive sequences as a strategy to amplify signal, enabled the first FISH-based genome-wide RNAi screen. Oligo FISH probes have also been generated directly from genomic DNA using many parallel PCR reactions.

The availability of complex oligo libraries produced by massively parallel synthesis has enabled a new generation of oligo-based technologies. These libraries are synthesized on a solid substrate, then amplified or chemically cleaved in order to move the library into solution. Popular applications of oligo libraries include targeted capture for next generation sequencing and custom gene synthesis. Two very recent studies have used complex libraries to visualize single-copy regions of mammalian genomes by FISH. One study used long oligos (>150 bp) as templates for PCR, and then labeled the amplification products non-specifically, while the other adapted a 75-100 bp single-stranded sequence-capture library for FISH by replacing the 5′ biotin with a fluorophore.

Additional labeled probes include those known as “oligopaints” as described in US 2010/0304994 hereby incorporated by reference in its entirety for all purposes. As used herein, the term “Oligopaint” refers to detectably labeled polynucleotides that have sequences complementary to an oligonucleotide sequence, e.g., a portion of a DNA sequence e.g., a particular chromosome or sub-chromosomal region of a particular chromosome. Oligopaints are generated from synthetic probes and arrays that are, optionally, computationally patterned (rather than using natural DNA sequences and/or chromosomes as a template). Since Oligopaints are generated using nucleic acid sequences that are present in a pool, they are no longer spatially addressable (i.e., no longer attached to an array). Surprisingly, however, this method increases resolution of the oligopaints over chromosome paints that are made using yeast artificial chromosomes (YACs), bacterial artificial chromosomes (BACs), and/or flow sorted chromosomes.

In certain exemplary embodiments, small Oligopaints are provided. As used herein, the term “small Oligopaint” refers to an Oligopaint of between about 5 bases and about 100 bases long, or an Oligopaint of about 5 bases, about 10 bases, about 15 bases, about 20 bases, about 25 bases, about 30 bases, about 35 bases, about 40 bases, about 45 bases, about 50 bases, about 55 bases, about 60 bases, about 65 bases, about 70 bases, about 75 bases, about 80 bases, about 85 bases, about 90 bases, about 95 bases, or about 100 bases. Small Oligopaints can access targets that are not accessible to longer oligonucleotide probes. For example, in certain aspects small Oligopaints can pass into a cell, can pass into a nucleus, and/or can hybridize with targets that are partially bound by one or more proteins, etc. Small Oligopaints are also useful for reducing background, as they can be more easily washed away than larger hybridized oligonucleotide sequences. As used herein, the terms “Oligopainted” and “Oligopainted region” refer to a target nucleotide sequence (e.g., a chromosome) or region of a target nucleotide sequence (e.g., a sub-chromosomal region), respectively, that has hybridized thereto one or more Oligopaints. Oligopaints can be used to label a target nucleotide sequence, e.g., chromosomes and sub-chromosomal regions of chromosomes during various phases of the cell cycle including, but not limited to, interphase, preprophase, prophase, prometaphase, metaphase, anaphase, telophase and cytokenesis.

Nucleic Acid

The terms “nucleic acid,” “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides or ribonucleotides, or analogs thereof. The labeled probes or anti-lock probes described herein may include or be a “nucleic acid,” “nucleic acid molecule,” “nucleic acid sequence,” “nucleic acid fragment,” “oligonucleotide” or “polynucleotide.” Oligonucleotides or polynucleotides useful in the methods described herein may comprise natural nucleic acid sequences and variants thereof, artificial nucleic acid sequences, or a combination of such sequences. Oligonucleotides or polynucleotides may be single stranded or double stranded.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

In certain exemplary embodiments, nucleotide analogs or derivatives will be used, such as nucleosides or nucleotides having protecting groups on either the base portion or sugar portion of the molecule, or having attached or incorporated labels, or isosteric replacements which result in monomers that behave in either a synthetic or physiological environment in a manner similar to the parent monomer. The nucleotides can have a protecting group which is linked to, and masks, a reactive group on the nucleotide. A variety of protecting groups are useful in the invention and can be selected. According to one aspect, self-avoiding nucleotides can be used to make labeled probes and anti-lock probes. Self-avoiding nucleotides are those which are capable of base pairing with natural nucleotides, but not with themselves. Self-avoiding nucleotides are known to those of skill in the art and are described in Hoshika, et al, Angew. Chem. Int. Ed. 2010, 49, pp. 5554-5557 and Hoshika et al., Nucleic Acids Research (2008) hereby incorporated by reference in their entireties.

Oligonucleotide sequences, such as single stranded oligonucleotide sequences to be used for labeled probes or anti-lock probes, may be isolated from natural sources, synthesized or purchased from commercial sources. In certain exemplary embodiments, oligonucleotide sequences may be prepared using one or more of the phosphoramidite linkers and/or sequencing by ligation methods known to those of skill in the art. Oligonucleotide sequences may also be prepared by any suitable method, e.g., standard phosphoramidite methods such as those described herein below as well as those described by Beaucage and Carruthers ((1981) Tetrahedron Lett. 22: 1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

In certain exemplary embodiments, oligonucleotide sequences may be prepared using a variety of microarray technologies known in the art. Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93:13555; Synthetic DNA Arrays In Genetic Engineering, Vol. 20:111, Plenum Press (1998); Duggan et al. (1999) Nat. Genet. S21:10; Microarrays: Making Them and Using Them In Microarray Bioinformatics, Cambridge University Press, 2003; U.S. Patent Application Publication Nos. 2003/0068633 and 2002/0081582; U.S. Pat. Nos. 6,833,450, 6,830,890, 6,824,866, 6,800,439, 6,375,903 and 5,700,637; and PCT Application Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597.

Polymerase recognition sites, cleavage sites and/or label or detectable moiety addition sites may be added to the single stranded oligonucleotides during synthesis using known materials and methods.

Oligonucleotide Probes

Oligonucleotide probes useful for labeled probes or anti-lock probes according to the present disclosure may have any desired nucleotide length and nucleic acid sequence. Accordingly, aspects of the present disclosure are directed to the use of a plurality or set of nucleic acid probes, such as single stranded nucleic acid probes, such as oligonucleotide paints. The term “probe” refers to a single-stranded oligonucleotide sequence that will recognize and form a hydrogen-bonded duplex with a complementary sequence in a target nucleic acid sequence or its cDNA derivative. The probe includes a target hybridizing nucleic acid sequence. Exemplary nucleic acid sequences may be short nucleic acids or long nucleic acids. Exemplary nucleic acid sequences include oligonucleotide paints. Exemplary nucleic acid sequences are those having between about 1 nucleotide to about 100,000 nucleotides, between about 3 nucleotides to about 50,000 nucleotides, between about 5 nucleotides to about 10,000 nucleotides, between about 10 nucleotides to about 10,000 nucleotides, between about 10 nucleotides to about 1,000 nucleotides, between about 10 nucleotides to about 500 nucleotide, between about 10 nucleotides to about 100 nucleotides, between about 10 nucleotides to about 70 nucleotides, between about 15 nucleotides to about 50 nucleotides, between about 20 nucleotides to about 60 nucleotides, between about 50 nucleotides to about 500 nucleotides, between about 70 nucleotides to about 300 nucleotides, between about 100 nucleotides to about 200 nucleotides, and all ranges or values in between whether overlapping or not. Exemplary oligonucleotide probes include between about 10 nucleotides to about 100 nucleotides, between about 10 nucleotides to about 70 nucleotides, between about 15 nucleotides to about 50 nucleotides, between about 20 nucleotides to about 60 nucleotides and all ranges and values in between whether overlapping or not. According to one aspect, oligonucleotide probes according to the present disclosure should be capable of hybridizing to a target nucleic acid. Probes according to the present disclosure may include a label or detectable moiety as described herein. Oligonucleotides or polynucleotides may be designed, if desired, with the aid of a computer program such as, for example, DNAWorks, or Gene2Oligo.

Oligonucleotide probes according to the present disclosure need not form a perfectly matched duplex with the single stranded nucleic acid, though a perfect matched duplex is exemplary. According to one aspect, oligonucleotide probes as described herein form a stable hybrid with that of the target sequence under stringent to moderately stringent hybridization and wash conditions. If it is expected that the probes will be essentially completely complementary (i.e., about 99% or greater) to the target sequence, stringent conditions will be used. If some mismatching is expected, with the result that the probe will not be completely complementary, the stringency of hybridization may be lessened. Conditions which affect hybridization, and which select against nonspecific binding are known in the art, and are described in, for example, Sambrook et al., (2001). Generally, lower salt concentration and higher temperature increase the stringency of binding. For example, it is usually considered that stringent conditions are incubations in solutions which contain approximately 0.1×SSC, 0.1% SDS, at about 65° C. incubation/wash temperature, and moderately stringent conditions are incubations in solutions which contain approximately 1-2×SSC, 0.1% SDS and about 50°-65° C. incubation/wash temperature. Low stringency conditions are 2×SSC and about 30°-50° C.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to exemplary conditions under which a probe or primer will hybridize to its target sequence, to a detectably greater degree than other sequences (e.g. at least 2-fold over background). Other such conditions may be appropriate. Stringent conditions are sequence dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength and pH) at which 50% of a complementary target sequence hybridizes to a perfectly matched probe or primer. Typically, stringent conditions will be those in which the salt concentration is less than about 1.0 M Na⁺ ion, typically about 0.01 to 1.0 M Na ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001. It is to be understood that any desired stringency and/or conditions may be employed as desired.

Nucleic acid probes according to the present disclosure may be labeled or unlabeled. Certain nucleic acid probes may be directly labeled or indirectly labeled.

According to certain aspects, nucleic acid probes may include a primary nucleic acid sequence that is non-hybridizable to a target nucleic acid sequence in addition to the sequence of the probe that hybridizes to the target nucleic acid sequence. Exemplary primary nucleic acid sequences or target non-hybridizing nucleic acid sequences include between about 10 nucleotides to about 100 nucleotides, between about 10 nucleotides to about 70 nucleotides, between about 15 nucleotides to about 50 nucleotides, between about 20 nucleotides to about 60 nucleotides and all ranges and values in between whether overlapping or not. According to certain aspects, the primary nucleic acid sequence is hybridizable with one or more secondary nucleic acid sequences. According to certain aspects, the secondary nucleic acid sequence may include a label. According to this aspect, the nucleic acid probes are indirectly labeled as the secondary nucleic acid binds to the primary nucleic acid thereby indirectly labeling the probe which hybridizes to the target nucleic acid sequence. According to certain aspects, a plurality of nucleic acid probes is provided with each having a common primary nucleic acid sequence. That is, the primary nucleic acid sequence is common to a plurality of nucleic acid probes, such that each nucleic acid probe in the plurality has the same or substantially similar primary nucleic acid sequence. According to one aspect, the primary nucleic acid sequence is a single sequence species. In this manner, a plurality of common secondary nucleic acid sequences is provided which hybridize to the plurality of common primary nucleic acid sequences. That is, each secondary nucleic acid sequence has the same or substantially similar nucleic acid sequence. According to one exemplary embodiment, a single primary nucleic acid sequence is provided for each of the nucleic acid probes in the plurality. Accordingly, only a single secondary nucleic acid sequence which is hybridizable to the primary nucleic acid sequence need be provided to label each of the nucleic acid probes. According to certain aspects, the common secondary nucleic acid sequences may include a common label. According to this aspect, a plurality of nucleic acid probes are provided having substantially diverse nucleic acid sequences hybridizable to different target nucleic acid sequences and where the plurality of nucleic acid probes have common primary nucleic acid sequences. Accordingly, a common secondary nucleic acid sequence having a label may be used to indirectly label each of the plurality of nucleic acid probes. According to this aspect, a single or common primary nucleic acid sequence and secondary nucleic acid sequence pair can be used to indirectly label diverse nucleic acid probe sequences. Such an embodiment is provided where a plurality of nucleic acid probes having primary nucleic acid sequences are commercially synthesized, such as on an array. Labeled secondary nucleic acid sequences can also be commercially synthesized so that they are hybridizable with the primary nucleic acid sequences. The nucleic acid probes may be combined with the labeled secondary nucleic acids and one or more or a plurality of target nucleic acid sequences under conditions such that the nucleic acid probe or probes hybridize to the target nucleic acid sequence or sequences while the primary nucleic acid sequence is nonhybridizable to the target nucleic acid sequence or sequences. A labeled secondary nucleic acid sequence hybridizes with a corresponding primary nucleic acid sequence to indirectly label the nucleic acid probe, thereby labeling the target nucleic acid sequence. According to one aspect, the nucleic acid probes may be combined with the labeled secondary nucleic acids and one or more or a plurality of target nucleic acid sequences together in a one pot method. According to one aspect, the nucleic acid probes may be combined with the labeled secondary nucleic acids and one or more or a plurality of target nucleic acid sequences sequentially, such as the nucleic acid probes are combined with the target nucleic acid to form a mixture and then the labeled secondary nucleic acid is combined with the mixture or the nucleic acid probes are combined with the labeled secondary nucleic acids to form a mixture and then the target nucleic acid is combined with the mixture.

According to certain aspects, the primary nucleic acid sequence is modifiable with one or more labels. According to this aspect, one or more labels may be added to the primary nucleic acid sequence using methods known to those of skill in the art.

According to an additional embodiment, nucleic acid probes may include a first half of a ligand-ligand binding pair, such as biotin-avidin. Such nucleic acid probes may or may not include a primary nucleic acid sequence. The first half of a ligand-ligand binding pair may be attached directly to the nucleic acid probe. According to certain aspects, a second half of the ligand-ligand binding pair may include a label. Accordingly, the nucleic acid probe may be indirectly labeled by the use of a ligand-ligand binding pair. According to certain aspects, a common ligand-ligand binding pair may be used with a plurality of nucleic acid probes of different nucleic acid sequences. Accordingly, a single species of ligand-ligand binding pair may be used to indirectly label a plurality of different nucleic acid probe sequences. The common ligand-ligand binding pair may include a common label or a plurality of common ligand-ligand binding pairs may be labeled with different labels. Accordingly, a plurality of nucleic acid probes of different nucleic acid sequences may be labeled with a single species of label using a single species of a ligand-ligand binding pair.

According to one aspect, the primary nucleic acid sequences may include one or more subsequences that are hybridizable with one or more different secondary nucleic sequences. The one or more secondary nucleic acid sequences may include one or more subsequences that hybridize with one or more tertiary nucleic acid sequences, and so on. Each of the primary nucleic acid sequences, the secondary nucleic acid sequences, the tertiary nucleic acid sequences and so on may be directly labeled with a label or may be indirectly labeled with a label. In this manner, an exponential labeling of the nucleic acid probe can be achieved.

Labels

A label according to the present disclosure includes a functional moiety directly or indirectly attached or conjugated to a nucleic acid which provides a desired function. According to certain aspects, a label may be used for detection. Detectable labels or moieties are known to those of skill in the art. According to certain aspects, a label may be used to retrieve a particular molecule. Retrievable labels or moieties are known to those of skill in the art. According to certain aspects, a label may be used to target a particular molecule to a target nucleic acid of interest for a desired function. Targeting labels or moieties are known to those of skill in the art. According to certain aspects, a label may be used to react with a target nucleic acid of interest. Reactive labels or moieties are known to those of skill in the art. According to certain aspects, a label may be an antibody, ligand, hapten, radioisotope, therapeutic agent and the like.

As used herein, the term “retrievable moiety” refers to a moiety that is present in or attached to a polynucleotide that can be used to retrieve a desired molecule or factors bound to a desired molecule (e.g., one or more factors bound to a targeting moiety). As used herein, the term “retrievable label” refers to a label that is attached to a polynucleotide (e.g., an Oligopaint) and can, optionally, be used to specifically and/or nonspecifically bind a target protein, peptide, DNA sequence, RNA sequence, carbohydrate or the like at or near the nucleotide sequence to which one or more Oligopaints have hybridized. In certain aspects, target proteins include, but are not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

As used herein, the term “targeting moiety” refers to a moiety that is present in or attached to a polynucleotide that can be used to specifically and/or nonspecifically bind one or more factors that associate with, modify or otherwise interact with a nucleic acid sequence of interest (e.g., DNA (e.g., nuclear, mitochondrial, transfected and the like) and/or RNA), including, but not limited to, a protein, a peptide, a DNA sequence, an RNA sequence, a carbohydrate, a lipid, a chemical moiety or the like at or near the nucleotide sequence of interest to which the polynucleotide has hybridized. In certain aspects, factors that associate with a nucleic acid sequence of interest include, but are not limited to histone proteins (e.g., H1, H2A, H2B, H3, H4 and the like, including monomers and oligomers (e.g., dimers, tetramers, octamers and the like)) scaffold proteins, transcription factors, DNA binding proteins, DNA repair factors, DNA modification proteins (e.g., acetylases, methylases and the like).

In other aspects, factors that associate with, modify or otherwise interact with a nucleic acid sequence of interest are proteins including, but not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) acetylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

In certain aspects, a targeting and/or retrievable moiety is activatable. As used herein, the term “activatable” refers to a targeting and/or retrievable moiety that is inert (i.e., does not bind a target) until activated (e.g., by exposure of the activatable, targeting and/or retrievable moiety to light, heat, one or more chemical compounds or the like). In other aspects, a targeting and/or retrievable moiety can bind one or more targets without the need for activation of the targeting and/or retrievable moiety. Exemplary methods for attaching proteins, lipids, carbohydrates, nucleic acids and the like are known to those of skill in the art. In certain aspects, a targeting moiety can be a non-targeting moiety that is cross-linked or otherwise modified to bind one or more factors that associate with, modify or otherwise interact with a nucleic acid sequence.

In certain exemplary embodiments, a targeting moiety, a retrievable moiety and/or polynucleotide has a detectable label bound thereto. As used herein, the term “detectable label” refers to a label that can be used to identify a target (e.g., a factor associated with a nucleic acid sequence of interest, a chromosome or a sub-chromosomal region). Typically, a detectable label is attached to the 3′- or 5′-end of a polynucleotide. Alternatively, a detectable label is attached to an internal portion of an oligonucleotide. Detectable labels may vary widely in size and compositions; the following references provide guidance for selecting oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like.

Methods for incorporating detectable labels into nucleic acid probes are well known. Typically, detectable labels (e.g., as hapten- or fluorochrome-conjugated deoxyribonucleotides) are incorporated into a nucleic acid, such as a nucleic acid probe during a polymerization or amplification step, e.g., by PCR, nick translation, random primer labeling, terminal transferase tailing (e.g., one or more labels can be added after cleavage of the primer sequence), and others (see Ausubel et al., 1997, Current Protocols In Molecular Biology, Greene Publishing and Wiley-Interscience, New York).

In certain aspects, a suitable targeting moiety, retrievable moiety or detectable label includes, but is not limited to, a capture moiety such as a hydrophobic compound, an oligonucleotide, an antibody or fragment of an antibody, a protein, a peptide, a chemical cross-linker, an intercalator, a molecular cage (e.g., within a cage or other structure, e.g., protein cages, fullerene cages, zeolite cages, photon cages, and the like), or one or more elements of a capture pair, e.g., biotin-avidin, biotin-streptavidin, NHS-ester and the like, a thioether linkage, static charge interactions, van der Waals forces and the like (See, e.g., Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160). In certain aspects, a suitable targeting label, retrievable label or detectable label is an enzyme (e.g., a methylase and/or a cleaving enzyme). In one aspect, an antibody specific against the enzyme can be used to retrieve or detect the enzyme and accordingly, retrieve or detect an oligonucleotide sequence or factor attached to the enzyme. In another aspect, an antibody specific against the enzyme can be used to retrieve or detect the enzyme and, after stringent washes, retrieve or detect a factor or first oligonucleotide sequence that is hybridized to a second oligonucleotide sequence having the enzyme attached thereto.

Biotin, or a derivative thereof, may be used as an oligonucleotide label (e.g., as a targeting moiety, retrievable moiety and/or a detectable label), and subsequently bound by a avidin/streptavidin derivative (e.g., detectably labelled, e.g., phycoerythrin-conjugated streptavidin), or an anti-biotin antibody (e.g., a detectably labelled antibody). Digoxigenin may be incorporated as a label and subsequently bound by a detectably labelled anti-digoxigenin antibody (e.g., a detectably labelled antibody, e.g., fluoresceinated anti-digoxigenin). An aminoallyl-dUTP residue may be incorporated into an oligonucleotide and subsequently coupled to an N-hydroxy succinimide (NHS) derivatized fluorescent dye. In general, any member of a conjugate pair may be incorporated into a retrievable moiety and/or a detectable label provided that a detectably labelled conjugate partner can be bound to permit detection. As used herein, the term antibody refers to an antibody molecule of any class, or any sub-fragment thereof, such as an Fab.

Other suitable labels (targeting moieties, retrievable moieties and/or detectable labels) include, but are not limited to, fluorescein (FAM), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for reaction, retrieval and/or detection: biotin/a-biotin, digoxigenin/a-digoxigenin, dinitrophenol (DNP)/α-DNP, 5-Carboxyfluorescein (FAM)/α-FAM.

Additional suitable labels (targeting moieties, retrievable moieties and/or detectable labels) include, but are not limited to, chemical cross-linking agents. Cross-linking agents typically contain at least two reactive groups that are reactive towards numerous groups, including, but not limited to, sulfhydryls and amines, and create chemical covalent bonds between two or more molecules. Functional groups that can be targeted with cross-linking agents include, but are not limited to, primary amines, carboxyls, sulfhydryls, carbohydrates and carboxylic acids. Protein molecules have many of these functional groups and therefore proteins and peptides can be readily conjugated using cross-linking agents. Cross-linking agents are well known in the art and are commercially available (Thermo Scientific (Rockford, Ill.)).

A detectable moiety, label or reporter can be used to detect a nucleic acid or nucleic acid probe as described herein. Oligonucleotide probes or nucleic acid probes described herein can be labeled in a variety of ways, including the direct or indirect attachment of a detectable moiety such as a fluorescent moiety, hapten, colorimetric moiety and the like. A location where a label may be attached is referred to herein as a label addition site or detectable moiety addition site and may include a nucleotide to which the label is capable of being attached. One of skill in the art can consult references directed to labeling DNA. Examples of detectable moieties include various radioactive moieties, enzymes, prosthetic groups, fluorescent markers, luminescent markers, bioluminescent markers, metal particles, protein-protein binding pairs, protein-antibody binding pairs and the like. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C, or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. Nos. 5,188,934 (4,7-dichlorofluorescein dyes); 5,366,860 (spectrally resolvable rhodamine dyes); 5,847,162 (4,7-dichlororhodamine dyes); 4,318,846 (ether-substituted fluorescein dyes); 5,800,996 (energy transfer dyes); Lee et al.; 5,066,580 (xanthine dyes); 5,688,648 (energy transfer dyes); and the like. Labeling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY TM 630/650-14-dUTP, BODIPY TM 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY ™ FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Alternatively, the above fluorophores and those mentioned herein may be added during oligonucleotide synthesis using for example phosphoroamidite or NHS chemistry. Protocols are known in the art for custom synthesis of nucleotides having other fluorophores (See, Henegariu et al. (2000) Nature Biotechnol. 18:345). 2-Aminopurine is a fluorescent base that can be incorporated directly in the oligonucleotide sequence during its synthesis. Nucleic acid could also be stained, a priori, with an intercalating dye such as DAPI, YOYO-1, ethidium bromide, cyanine dyes (e.g. SYBR Green) and the like.

Other fluorophores available for post-synthetic attachment include, but are not limited to, ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red (available from Molecular Probes, Inc., Eugene, Oreg.), Cy2, Cy3, Cy3.5, Cy5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver or gold particles may be used to enhance signal from fluorescently labeled nucleotide and/or oligonucleotide sequences (Lakowicz et al. (2003) BioTechniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Biotin/avidin is an example of a ligand-ligand binding pair. An antibody/antigen binging pair may also be used with methods described herein. Other ligand-ligand binding pairs or conjugate binding pairs are well known to those of skill in the art. Digoxigenin may be incorporated as a label and subsequently bound by a detectably labeled anti-digoxigenin antibody (e.g. fluoresceinated anti-digoxigenin). An aminoallyl-dUTP or aminohexylacrylamide-dCTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM, FITC), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

According to certain aspects, detectable moieties described herein are spectrally resolvable. “Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

In certain embodiments, the detectable moieties can provide higher detectability when used with an electron microscope, compared with common nucleic acids. Moieties with higher detectability are often in the group of metals and organometals, such as mercuric acetate, platinum dimethylsulfoxide, several metal-bipyridyl complexes (e.g. osmium-bipy, ruthenium-bipy, platinum-bipy). While some of these moieties can readily stain nucleic acids specifically, linkers can also be used to attach these moieties to a nucleic acid. Such linkers added to nucleotides during synthesis are acrydite- and a thiol-modified entities, amine reactive groups, and azide and alkyne groups for performing click chemistry. Some nucleic acid analogs are also more detectable such as gamma-adenosine-thiotriphosphate, iododeoxycytidine-triphosphate, and metallonucleosides in general (see Dale et al., Proc. Nat. Acad. Sci. USA, Vol. 70, No. 8, pp. 2238-2242 (1973)). The modified nucleotides are added during synthesis. Synthesis may refer by example to solid support synthesis of oligonucleotides. In this case, modified nucleic acids, which can be a nucleic acid analog, or a nucleic acid modified with a detectable moiety, or with an attachment chemistry linker, are added one after each other to the nucleic acid fragments being formed on the solid support, with synthesis by phosphoramidite being the most popular method. Synthesis may also refer to the process performed by a polymerase while it synthesizes the complementary strands of a nucleic acid template. Certain DNA polymerases are capable of using and incorporating nucleic acids analogs, or modified nucleic acids, either modified with a detectable moiety or an attachment chemistry linker to the complementary nucleic acid template.

Detection method(s) used will depend on the particular detectable labels used in the reactive labels, retrievable labels and/or detectable labels. In certain exemplary embodiments, target nucleic acids such as chromosomes and sub-chromosomal regions of chromosomes during various phases of the cell cycle including, but not limited to, interphase, preprophase, prophase, prometaphase, metaphase, anaphase, telophase and cytokinesis, having one or more reactive labels, retrievable labels, or detectable labels bound thereto by way of the probes described herein may be selected for and/or screened for using a microscope, a spectrophotometer, a tube luminometer or plate luminometer, x-ray film, a scintillator, a fluorescence activated cell sorting (FACS) apparatus, a microfluidics apparatus or the like.

When fluorescently labeled targeting moieties, retrievable moieties, or detectable labels are used, fluorescence photomicroscopy can be used to detect and record the results of in situ hybridization using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

In certain exemplary embodiments, images of fluorescently labeled chromosomes are detected and recorded using a computerized imaging system such as the Applied Imaging Corporation CytoVision System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al., supra.

In situ hybridization methods using probes described herein can be performed on a variety of biological or clinical samples, in cells that are in any (or all) stage(s) of the cell cycle (e.g., mitosis, meiosis, interphase, G0, G1, S and/or G2). Examples include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. Samples are prepared for assays of the invention using conventional techniques, which typically depend on the source from which a sample or specimen is taken. These examples are not to be construed as limiting the sample types applicable to the methods and/or compositions described herein.

In certain exemplary embodiments, probes include multiple chromosome-specific probes, which are differentially labeled (i.e., at least two of the chromosome-specific probes are differently labeled). Various approaches to multi-color chromosome painting have been described in the art and can be adapted to the present invention following the guidance provided herein. Examples of such differential labeling (“multicolor FISH”) include those described by Schrock et al. (1996) Science 273:494, and Speicher et al. (1996) Nature Genet. 12:368). Schrock et al. describes a spectral imaging method, in which epifluorescence filter sets and computer software is used to detect and discriminate between multiple differently labeled DNA probes hybridized simultaneously to a target chromosome set. Speicher et al. describes using different combinations of 5 fluorochromes to label each of the human chromosomes (or chromosome arms) in a 27-color FISH termed “combinatorial multifluor FISH”). Other suitable methods may also be used (see, e.g., Ried et al., 1992, Proc. Natl. Acad. Sci. USA 89:1388-92).

Hybridization of the labeled probes and the anti-lock probes described herein to target chromosomes sequences can be accomplished by standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides (e.g., hybridized Oligopaints). The reagents used in each of these steps and their conditions of use vary depending on the particular situation and whether their use is required with any particular probes. Hybridization conditions are also described in U.S. Pat. No. 5,447,841. It will be appreciated that numerous variations of in situ hybridization protocols and conditions are known and may be used in conjunction with the present invention by practitioners following the guidance provided herein.

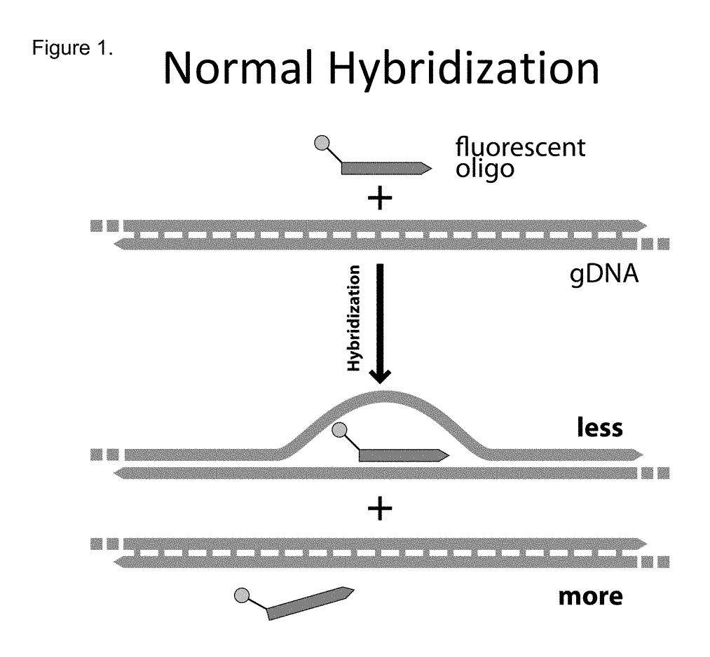

FIG. 1 illustrates a typical hybridization process where, in fixed chromatin, the two strands of genomic DNA remain in proximity even after denaturation. When the fluorescent oligo probe is combined with the genomic double stranded DNA in the state of having a portion separated into a first single strand segment and a complementary single strand segment, some of the probe hybridizes to the first single strand segment while some of the probe fails to hybridize. The efficiency of hybridization is reflected in the percent of the probe that hybridizes. FIG. 1 depicts a situation where the hybridization efficiency is low. During hybridization, the two genomic DNA strands are thermodynamically and kinetically favored to re-anneal and block binding of a labeled probe. In additional, where labeled probes have already bound to their target loci before genomic strands re-anneal, the separate genomic strands can isothermally displace the bound probes using branch-migration mechanisms. Accordingly, a low hybridization efficiency results in reduced signal intensity, continuity, consistency among samples, sensitivity, etc.

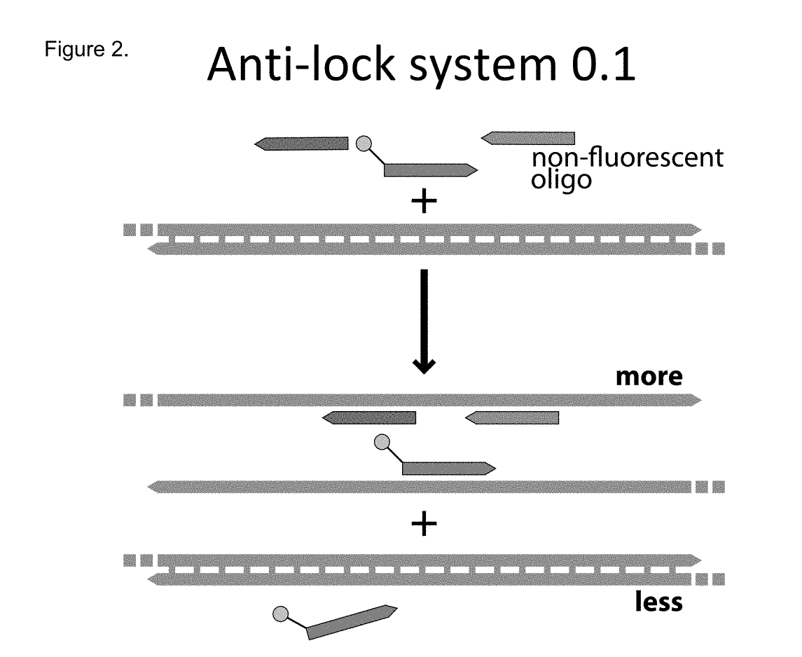

FIG. 2 depicts a system of probes including a labeled probe (with the label shown) and two anti-lock probes (with no label shown). The anti-lock probes are shown as lacking a label, however embodiments of the present disclosure contemplate an anti-lock probe having a label. The labeled probe is complementary to a first single strand segment and the two anti-lock probes are complementary to the complementary single strand segment. Accordingly, when the labeled probe and the anti-lock probes are combined with the genomic DNA in the state of having a portion separated into a first single strand segment and a complementary single strand segment, the labeled probe binds to the first single strand segment and the anti-lock probes bind to the complementary single strand segment. Because the anti-lock probes are bound to the complementary single strand, re-annealing of the first single strand segment and the complementary single strand segment is inhibited. According to this aspect, the binding efficiency of the labeled probe is increased because, it is believed, the inhibition of re-annealing allows the labeled probe to bind to its target and remain bound.

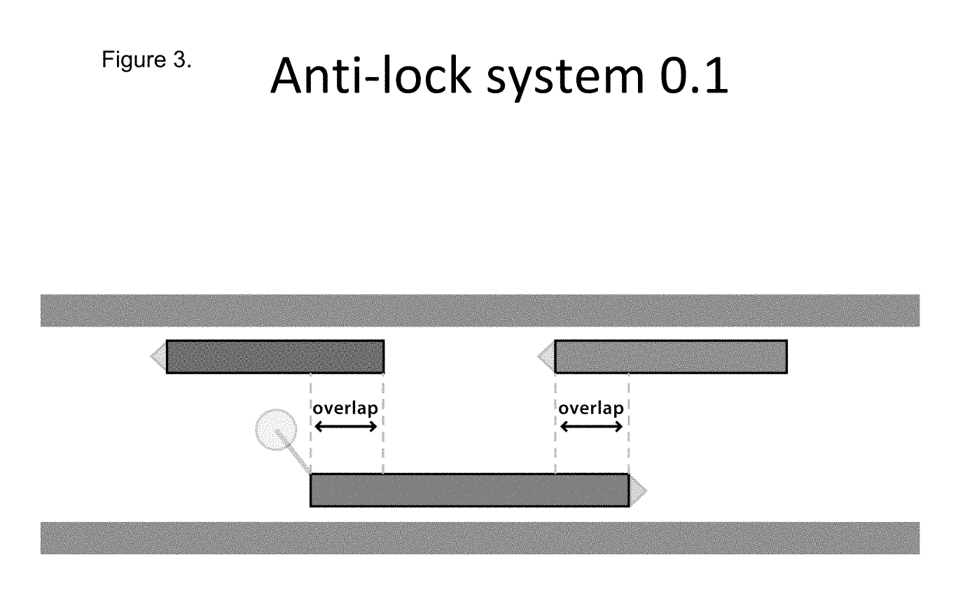

According to one aspect as depicted in FIG. 2 and FIG. 3, the anti-lock probes may partially overlap the labeled probe. This means that an anti-lock probe may bind to a portion of the sequence complementary to the sequence bound by the labeled probe on the first single strand segment. It is believed that the partial overlap enhances the binding of the labeled probe to the genomic DNA first single strand segment. It is believed that the partially overlapping anti-lock probes provide the labeled probe a steric advantage by inhibiting re-annealing, i.e., keeping the separated strand portion of the genomic DNA separated or “open” and also provide a thermodynamic advantage by locally reducing the Tm of the genomic DNA at the target site without reducing the Tm of the labeled probe for its target. The overlap also inhibits the separated strand portion of the genomic DNA from displacing the labeled probe through isothermal branch migration mechanisms. According to one aspect, the overlap may be between about 1 nucleotide to about 10 nucleotides, such that the Tm of the overlap remains below the Tm of the labeled probe for its target. According to one aspect, the overlap may be between about 1 nucleotide to about 5 nucleotides, such that the Tm of the overlap remains below the Tm of the labeled probe for its target. According to one aspect, the binding of the anti-lock probes and the labeled probes is cooperative. According to one aspect, the anti-lock probes and the labeled probes mutually protect each other from displacement by the genomic DNA strands, through, for example, branch-migration. According to one aspect, the anti-lock probes may be of any suitable length when no label is attached to an antilock probe. According to one aspect, one of skill can select suitable concentrations of labeled probes, suitable concentrations of anti-lock probes, and suitable annealing temperatures and other hybridization conditions to achieve quantitative binding of the labeled probes to their targets. According to one aspect, quantitative binding of the labeled probes to their targets is greater than 50%, greater than 60%, greater than 70%, greater than 80%, greater than 90%, greater than 95%, greater than 96%, greater than 97%, greater than 98% or greater than 99%.

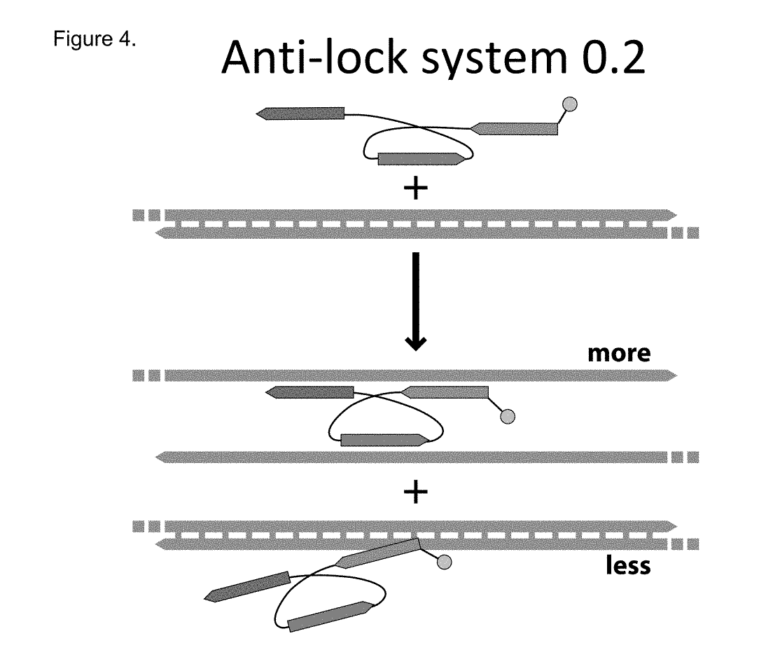

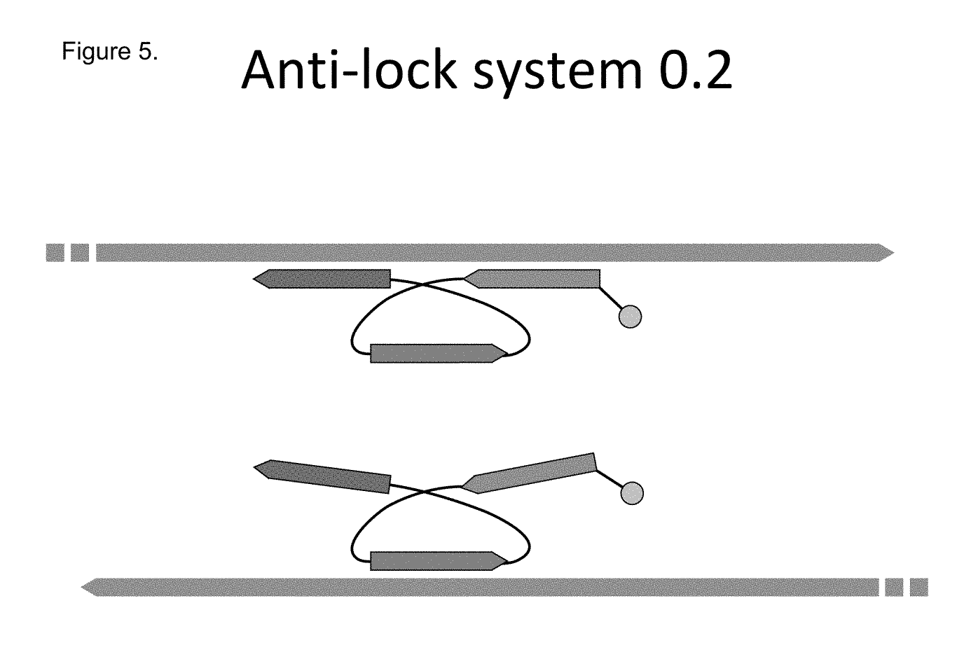

As depicted in FIG. 4, an anti-lock probe may be connected to a labeled probe to result in a linked probe. In FIG. 4, a labeled probe is connected to an antilock probe which is further connected to an anti-lock probe to result in a linked probe. The labeled probe hybridizes to a first single strand segment. An antilock probe hybridizes to the complementary single strand segment. An antilock probe hybridizes to the first single strand segment. The probes may be interconnected in series by nucleic acid sequences to produce a single polynucleotide linked probe having a labeled probe portion and one or more antilock probe portions. The interconnecting nucleic acid sequences are non-hybridizable to the genomic DNA. According to one aspect, the probes may be interconnected by linkage groups known to those of skill in the art. According to one aspect, antilock probes and labeled probes may be interconnected in any sequence as desired. For example, the labeled probe may be between antilock probes with the label being internal to the labeled probe. The combination of one or more antilock probes and a labeled probe interconnected by nucleotides or other linkage group, i.e. a linked probe, provides advantageous hybridization so as to increase efficiency of hybridization of the labeled probe in the manner described herein and also increases cooperativity of the binding of the labeled probe and the anti-lock probes. According to an alternate embodiment depicted in FIG. 5, two separate linked probes may be used to hybridize to each of the first single strand segment and the complementary single strand segment as each linked probe is capable of doing so because of the presence of a labeled probe and anti-lock probes as described herein. According to this aspect, both the first single strand segment and the complementary single strand segment can be labeled by the same species of linked probe.

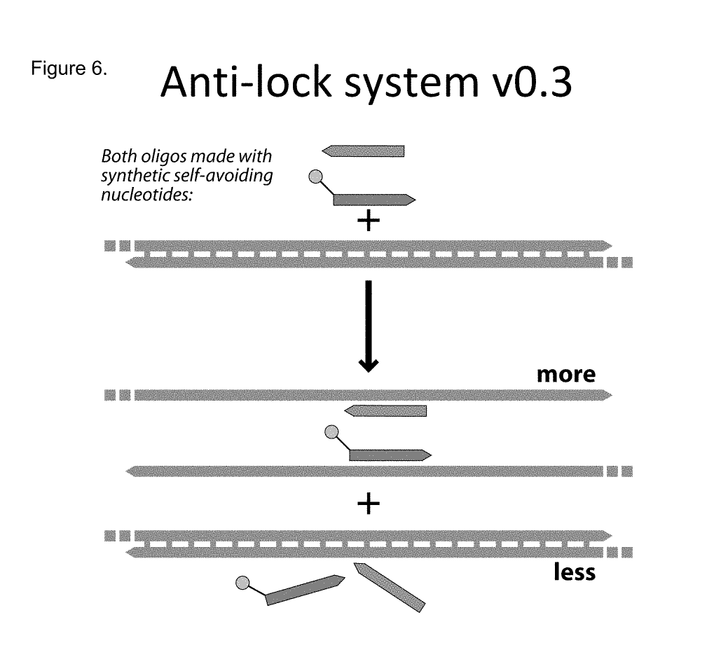

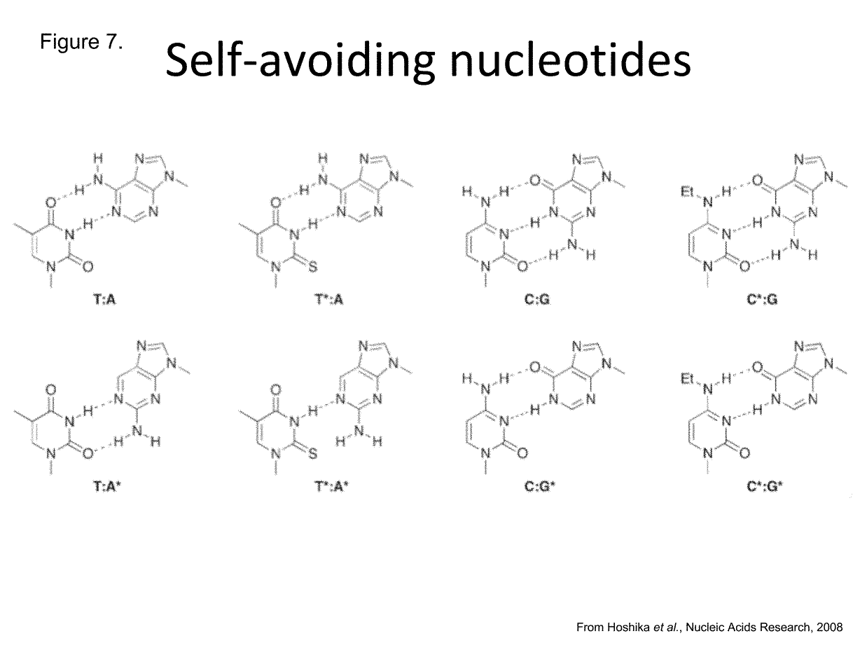

As depicted in FIG. 6, a labeled probe and an anti-lock probe may include self-avoiding nucleotides. Self-avoiding nucleotides are known to those of skill in the art and are capable of base pairing with natural nucleotides but they cannot base pair to themselves. According to this aspect, the sequence of the antilock-probe may be substantially or entirely complementary to the labeled probe thereby allowing the anti-lock probe to be completely overlapping with the labeled probe. Exemplary self-avoiding nucleotides are depicted in FIG. 7.

The contents of all references, patents and published patent applications cited throughout this application are hereby incorporated by reference in their entirety for all purposes.

### EQUIVALENTS

Other embodiments will be evident to those of skill in the art. It should be understood that the foregoing description is provided for clarity only and is merely exemplary. The spirit and scope of the present invention are not limited to the above example, but are encompassed by the claims. All publications, patents and patent applications cited above are incorporated by reference herein in their entirety for all purposes to the same extent as if each individual publication or patent application were specifically indicated to be so incorporated by reference.

What is claimed is:

1. A method of improving binding efficiency of a labeled probe to double stranded DNA having a portion of the double stranded DNA separated into a first single strand segment and a complementary single strand segment comprising
combining the double stranded DNA with a labeled probe that is complementary to the first single strand segment at a target sequence and one or more anti-lock probes that are complementary to either the first single strand segment or the complementary single strand segment wherein the labeled probe binds to the first single strand segment at the target sequence and the one or more anti-lock probes bind to at least the complementary single strand segment.

2. The method of claim 1 wherein the double stranded DNA is genomic DNA.

3. The method of claim 1 wherein the bound one or more anti-lock probes inhibits re-annealing of the first single strand segment and the complementary single strand segment.

4. The method of claim 1 wherein the labeled probe is between 2 nucleotides and 200 nucleotides in length.

5. The method of claim 1 wherein the labeled probe is an oligonucleotide paint.

6. The method of claim 1 wherein a first anti-lock probe binds to the complementary single strand segment at a position which overlaps with the bound labeled probe.

7. The method of claim 1 wherein a first anti-lock probe binds to the complementary single strand segment at a position which overlaps with the region complementary to the target sequence of the bound labeled probe.

8. The method of claim 1 wherein one or more anti-lock probes bind to the complementary single stranded segment at a position neighboring the region complementary to the target sequence of the bound labeled probe without overlap.

9. The method of claim 1 wherein a first anti-lock probe binds to the complementary single stranded segment at a position which overlaps with the region complementary to the target sequence of the bound labeled probe by at least one nucleotide.

10. The method of claim 1 wherein a first anti-lock probe binds to the complementary single strand segment at a position which overlaps with the bound labeled probe by between about 1 nucleotide and about 10 nucleotides.

11. The method of claim 1 wherein a first anti-lock probe binds to the complementary single strand segment at a position which overlaps with the region complementary to the target sequence of bound labeled probe by between about 1 nucleotide and about 10 nucleotides.

12. The method of claim 1 wherein a first anti-lock probe binds to the complementary single strand segment at a position which overlaps with the bound labeled probe by between about 1 nucleotide and about 5 nucleotides.

13. The method of claim 1 wherein a first anti-lock probe binds to the complementary single strand segment at a position which overlaps with the region complementary to the target sequence of the bound labeled probe by between about 1 nucleotide and about 5 nucleotides.

14. The method of claim 1 wherein a first anti-lock probe binds to the complementary single strand segment at a position which overlaps with the region complementary to the target sequence of the bound labeled probe and a second anti-lock probe binds to the complementary single strand segment at a position which overlaps with the region complementary to the target sequence of the bound labeled probe by at least 1 nucleotide.

15. The method of claim 1 wherein a first anti-lock probe binds to the complementary single strand segment at a position which overlaps with the bound labeled probe and a second anti-lock probe binds to the complementary single strand segment at a position which overlaps with the bound labeled probe by between about 1 nucleotide and about 10 nucleotides.

16. The method of claim 1 wherein a first anti-lock probe binds to the complementary single strand segment at a position which overlaps with the region complementary to the target sequence of the bound labeled probe and a second anti-lock probe binds to the complementary single strand segment at a position which overlaps with the region complementary to the target sequence of the bound labeled probe by between about 1 nucleotide and about 10 nucleotides.

17. The method of claim 1 wherein a first anti-lock probe binds to the complementary single strand segment at a position which overlaps with the bound labeled probe and a second anti-lock probe binds to the complementary single strand segment at a position which overlaps with the bound labeled probe by between about 1 nucleotide and about 5 nucleotides.

18. The method of claim 1 wherein a first anti-lock probe binds to the complementary single strand segment at a position which overlaps with the region complementary to the target sequence of the bound labeled probe and a second anti-lock probe binds to the complementary single strand segment at a position which overlaps with the region complementary to the target sequence of the bound labeled probe by between about 1 nucleotide and about 5 nucleotides.

19. The method of claim 1 wherein a first anti-lock probe binds to the complementary single stranded segment at a position which overlaps with the bound labeled probe and a second anti-lock probe binds to the first single stranded segment at a position which overlaps with the first antilock probe.

20. The method of claim 1 wherein a first anti-lock probe binds to the complementary single stranded segment at a position which overlaps with the region complementary to the target sequence of the bound labeled probe and a second anti-lock probe binds to the first single stranded segment at a position which overlaps with the region complementary to the target sequence of the first antilock probe.

21. The method of claim 1 wherein a first anti-lock probe binds to the complementary single strand segment at a position which overlaps with the bound labeled probe and a second anti-lock probe binds to the first single strand segment at a position which overlaps with the first antilock probe by between about 1 nucleotide and about 10 nucleotides.

22. The method of claim 1 wherein a first anti-lock probe binds to the complementary single strand segment at a position which overlaps with the bound labeled probe and a second anti-lock probe binds to the first single strand segment at a position which overlaps with the first antilock probe by between about 1 nucleotide and about 5 nucleotides.

23. The method of claim 1 wherein a first anti-lock probe binds to the complementary single stranded segment at a position which overlaps with the region complementary to the target sequence of the bound labeled probe and a second anti-lock probe binds to the first single stranded segment at a position which overlaps with the region complementary to the target sequence of the first antilock probe by between about 1 nucleotide and about 5 nucleotides.

24. The method of claim 1 wherein the labeled probe and one or more anti-lock probes are connected, creating a single molecule comprising the labeled probe and the anti-lock probes.

25. The method of claim 1 wherein the labeled probe and the one or more anti-lock probes are connected by one or more connector nucleotides.

26. The method of claim 1 wherein the labeled probe and the one or more anti-lock probes are connected in series by one or more connector nucleotides to form a continuous oligonucleotide strand.

27. The method of claim 1 wherein the labeled probe and the two or more anti-lock probes are connected in series by one or more connector nucleotides to form a continuous oligonucleotide strand with the labeled probe being at one end of the continuous oligonucleotide strand or between two or more anti-lock probes.

28. The method of claim 1 wherein the labeled probe and the one or more anti-lock probes are connected in series by one or more connector nucleotides to form a continuous oligonucleotide strand with the labeled probe being at one end of the continuous oligonucleotide strand and with a first anti-lock probe being hybridized to the first single strand segment at a complementary single stranded segment.

29. The method of claim 1 wherein the labeled probe and the one or more anti-lock probes are connected in series by one or more connector nucleotides to form a continuous oligonucleotide strand with the labeled probe being at one end of the continuous oligonucleotide strand or between two or more antilock probes and with a first anti-lock probe being hybridized to the complementary single strand segment.

30. The method of claim 1 wherein the labeled probe and the one or more anti-lock probes are connected in series by one or more connector nucleotides to form a continuous oligonucleotide strand with the labeled probe being at one end of the continuous oligonucleotide strand and with a first anti-lock probe being hybridized to the first single strand segment and a second antilock probe being hybridized to the complementary single strand segment.

31. The method of claim 1 wherein the labeled probe and the one or more anti-lock probes are connected by one or more connector nucleotides wherein the one or more connector nucleotides are unhybridizable to the first single strand segment or the complementary single strand segment.

32. The method of claim 1 wherein the labeled probe and the one or more anti-lock probes are connected by linker portions.

33. The method of claim 1 wherein the labeled probe and the one or more anti-lock probes include one or more of self-avoiding nucleotide analogues.

34. The method of claim 1 wherein the labeled probe and the one or more anti-lock probes include one or more of self-avoiding nucleotide analogues such that the labeled probe and the one or more anti-lock probes do not hybridize to each other.

35. The method of claim 1 wherein the labeled probe and a first anti-lock probe include one or more of self-avoiding nucleotide analogues such that the labeled probe and the one or more anti-lock probes are complementary sequences that do not hybridize to each other.

36. The method of claim 1 wherein the labeled probe and the one or more anti-lock probe are hybridized to target genomic DNA simultaneously.

37. The method of claim 1 wherein the one or more anti-lock probe is hybridized to genomic DNA first followed by the hybridization of the labeled probe.
